(19) United States
(12) Patent Application Publication
(10) Pub. No.: US 2019/0233820 A1
(43) Pub. Date: Aug. 1, 2019

(54) NANOPARTICLES FUNCTIONALIZED WITH GENE EDITING TOOLS AND RELATED METHODS

(71) Applicant: STEMGENICS, INC., Seattle, WA (US)

(72) Inventor: Andranik Andrew Aprikyan, Seattle, WA (US)

(73) Assignee: STEMGENICS, INC., Seattle, WA (US)

(21) Appl. No.: 16/341,025

(22) PCT Filed: Oct. 11, 2017
(86) PCT No.: PCT/US2017/056188

§ 371 Date: Apr. 10, 2019

Related U.S. Application Data
(60) Provisional application No. 62/406,542, filed on Oct. 11, 2016.

(51) Int. Cl. C12N 15/113; C12N 9/22; A61K 48/00; A61K 47/69

(52) U.S. Cl. CPC C12N 15/113; C12N 9/22; C12N 2310/20; A61K 47/6923; A61K 47/6937; A61K 48/0083

(57) ABSTRACT

This disclosure relates to compositions and methods for editing or altering target nucleotide sequences based on nanoparticle delivery vehicles. The compositions and methods can be applied to influence the functional expression of target gene products encoded by DNA and/or RNA. In some embodiments, the altered gene sequences are useful to normalize and regulate the function of target cells.

### CROSS-REFERENCE TO RELATED APPLICATION

This application claims the benefit of U.S. Provisional Application No. 62/406542, filed Oct. 11, 2016, the entire disclosure of which is expressly incorporated herein by reference.

### STATEMENT OF GOVERNMENT LICENSE RIGHTS

This invention was made with Government support under Small Business innovation Research (SBIR) Phase I IIP-1214943 awarded by the National Science Foundation. The Government has certain rights in the invention.

### FIELD OF THE INVENTION

This disclosure relates to methods and compositions for generating functionalized nanoparticles that alters nucleotide sequence and/or expression of target gene products encoded by DNA and/or RNA. The altered gene sequences are useful to normalize and regulate the function of target cells.

### BACKGROUND

Most of human diseases are due to inherited or acquired mutations in cell genome. Such mutations can be small including single nucleotide substitution causing amino acid substitution or premature termination for the gene expression, or larger such as insertion or deletion of larger segments consisting of two or more nucleotides. The affected area may include not only a gene coding sequence, but also regulatory sequences located prior to or after the coding areas. Recent technological advances with development of TALENs or CRISPR/Cas9 systems have made gene editing and mutational corrections possible.

The ability of cells with abnormal gene sequence to normally proliferate, migrate and differentiate to various cell types is altered in various pathological conditions, but it can be normalized upon mutational correction using gene editing tools. For example, abnormal cellular functions such as impaired survival and/or differentiation of bone marrow stem/progenitor cells into neutrophils are observed in patients with cyclic or severe congenital neutropenia who may have mutant neutrophil elastase gene, suffer from severe life-threatening infections and may evolve to develop acute myelogenous leukemia or other malignancies (Carlsson et al., Blood, 103, 3355 (2004); Carlsson et al., Haematologica, (91, 589 (2006)). Another example is Barth syndrome where patients may have abnormal survival of hematopoietic cells as well as impaired cardiac function called cardiomyopathy (Makaryan et al., Eur. J. Haematol., 88, 195 (2012), Aprikyan and Khuchua, Br. J Haematol, 161, 330 (2013)). Other inherited diseases like Barth syndrome, a multi-system stem cell disorder induced by presumably loss-of-function mutations in the mitochondrial TAZ gene, may be associated with neutropenia (reduced levels of blood neutrophils) that may cause recurring severe and sometimes life-threatening fatal infections and/or cardiomyopathy that may lead to heart failure that could be resolved by heart transplantation. The clinical abnormalities in such patients are triggered by specific mutations in different genes, which result in alteration of gene function and subsequent aberrant intracellular abnormalities leading to cell death or functional failure of the cells.

Treatment of neutropenic patients with granulocyte colony-stimulating factor (G-CSF) induces conformational changes in the G-CSF receptor molecule located on the cell surface, which subsequently triggers a chain of intracellular events that eventually restores the production of neutrophils to near normal level and improves the quality of life of the patients (Welte and Dale, Ann. Hematol. 72, 158 (1996)). Nevertheless, patients treated with G-CSF may evolve to develop leukemia (Aprikyan et al., Exp. Hematol 31, 372 (2003); Rosenberg et al., Br. J. Haematol. 140, 210 (2008); Newburger et al., Genes, Pediatr. Blood Cancer, 55, 314 (2010)), which is why alternative cell therapy approaches are being explored such as bone marrow or hematopoietic stem cell transplantation for treatment of neutropenia or ex vivo generation of cardiac cells upon differentiation of human induced pluripotent stem cells followed by transplantation of the newly generated cardiac cells into the patients' heart to fight heart failure and restore or improve cardiac muscle function (Makaryan et al., J Leukoc. Biol., 102, 1143 (2017).

An alternative molecular therapy approach includes gene editing tools such as TALENs and CRISPR/Cas systems represented by Class I CRISPR/Cas9 and Class II CRISPR/Cpfl systems (Gaj et al., Trends Biotechnol, 31, 397 (2013); Dong et al., Nature, 532, 522 (2017)). These technologies are based on the use of RNA or DNA molecules that guide gene cutting enzyme of choice, such as Cas9, nickase, Cpfl or other nucleases, to the specific sequence of interest. Such targeting creates single or double strand nucleotide sequence cut which can be repaired intracellularly, and if a donor nucleotide sequence is present with homology to the target-surrounding area, then homologous recombination occurs with insertion of the donor sequence that contains corrected nucleotides, thus resulting in gene editing and restoration of normal gene and cell function.

In general, gene-editing technology based on Zink-fingers, TALENS, and CRISPR-Cas9/Cpfl methodologies are characterized by low editing efficiency, off-target site cleavages that result in perturbation of cell genome integrity and may lead to various detrimental consequences, and inefficient delivery of gene-editing tools into the target cells. The present disclosure describes the development of a simple and reliable gene-editing technology that is somewhat comparable but yet distinct from the CRISPR-Cas approach that outperforms other methods and resolves the abovementioned problematic issues. Here a cell-permeable multi-functionalized nanoparticle is used as a single device with covalently linked bioactive molecules that penetrate through the cell membrane with high efficiency, reach the nucleus, bind the target gene of interest with high specificity, and introduce the gene-editing modifications. This nanoparticle-mediated gene editing is the most efficient driver for genome editing compared to alternative methods as it presents fast and robust introduction of gene editing tools into mammalian cells, minimizes the use of exogenous DNA capable of integrating and disrupting the target cell genome integrity and ensures highest gene-targeting specificity.

Currently, the multi-component gene editing tool utilizes guiding molecules that can be represented as RNA or DNA and DNA-cutting enzymes (nuclease, nickase) that are used either separately or with plasmid or lentiviral vectors for expression of guiding RNA/DNA and DNA-cutting enzymes. The use of such abundant DNA-containing system represents a major problem because such viral or plasmid delivery of different gene editing components is associated with random integration of DNA molecules into the cell genome, which is known to induce various mutations, alter normal gene expression pattern in the host cells, and trigger oncogene expression, thereby leading to cancer or other detrimental consequences. Furthermore, the nucleotide sequences from the viral and plasmid constructs may bind off-target sequences and therefore create new additional abnormal off-site alterations in otherwise normal cell genome. Therefore, the viral or plasmid based gene editing is not the best approach for nucleotide sequence manipulations and subsequent use in humans.

Furthermore, introduction of such guiding molecules and nuclease coding sequences into the cells are based on the use of electroporation or liposome-based fusion and with subsequent delivery of these molecules inside the cells. Both these approaches have problems associated with increased cell death and/or low transfection/delivery efficiency in various human cell types.

The present disclosure addresses the abovementioned concerns providing new alternatives for nucleotide sequence manipulation. Such gene editing tools can be safer and can more effectively correct and regulate normal gene function upon intracellular delivery of a cocktail of gene editing elements using distinctly non-integrating functionalized nanoparticles. Although the cellular membrane serves as an active barrier preserving the cascade of intracellular events from being affected by exogenous stimuli, these bioactive functionalized nanoparticles can penetrate cellular membranes to deliver gene editing elements to normalize, turn on or turn off expression of various genes of interest and/or control the cellular function, eliminate unwanted cells when needed, and/or directly reprogram human somatic cells into other cell types of interest.

Despite the advances in the art, a need remains for a more efficient approach to deliver biologically active molecules into the interior of a cell to efficiently induce genome editing of the cell while avoiding damage to the chromosomal structure integrity. The present disclosure fulfills the needs for non-integrative gene editing tools, minimization/elimination of the off-site targets, and preservation of intact human cell genome and provides new means to achieve further advantages related to controlled editing of a target gene sequence and/or its expression.

### SUMMARY

The present disclosure in some embodiments is directed to functionalization methods of linking proteins, peptides, DNA, RNA and/or other small molecules to biocompatible nanoparticles for genome correction and modulation of cellular functions. In some embodiments, the present disclosure is directed to the functionalized biocompatible nanoparticles themselves.

In one aspect, the disclosure provides a composition comprising a guide nucleic acid specific for a target nucleic acid sequence, a nuclease that modifies and/or cleaves the target nucleic acid sequence upon binding of the guide nucleic acid to the target nucleic acid sequence, and a nanoparticle. In some embodiments, the composition further comprises a donor nucleic acid molecule comprising a nucleic acid sequence for insertion into the cleavage site of the target nucleic acid sequence. The at least one of the guide nucleic acid and the nuclease is conjugated to the at least one nanoparticle.

In another aspect, the disclosure provides a cell that comprises the nanoparticle-based composition described herein.

In another aspect, the disclosure provides a method of altering a genome of a cell. The method comprises contacting a cell with a composition as described herein.

In another aspect, the disclosure provides a method of altering a genome or transcript of a cell. The method comprises contacting the cell with one or more functionalized nanoparticles. The one or more functional nanoparticles are conjugated to:

a guide nucleic acid specific for a target nucleic acid sequence in the genome or transcript, and

a protein capable of modifying the target nucleic acid sequence upon binding of the guide nucleic acid to the target nucleic acid sequence.

In some embodiments, the one or more of the nanoparticles is conjugated to a donor nucleic acid molecule comprising a nucleic acid sequence for insertion into the cleavage site of the target nucleic acid sequence.

These and other aspects of the present disclosure will become more readily apparent to those possessing ordinary skill in the art when reference is made to the following detailed description in conjunction with the accompanying drawings.

### DETAILED DESCRIPTION

In order to deliver biologically active molecules intracellularly, the present disclosure provides a universal platform based on a composition including a cell membrane-penetrating nanoparticle with covalently linked biologically active molecules. To this end, presented herein is a functionalization method that ensures a covalent linkage of proteins, peptides, DNA and/or RNA molecules to nanoparticles. The modified cell-permeable nanoparticles of the present disclosure provide a universal mechanism for intracellular delivery of biologically active molecules for regulation and/or normalization of cellular function in general, and editing nucleotide sequences to correct or improve gene expression and function, which can be subsequently used in research and development, drug screening and therapeutic applications to improve cellular function in humans.

The methods disclosed herein utilize biocompatible nanoparticles, including (but not limited to) for example, superparamagnetic iron oxide or gold nanoparticles, or polymeric nanoparticles further modified or otherwise similar to those previously described in scientific literature (e.g., Lewin et al., Nat. Biotech. 18, 410-414, (2000); Shen et al., Magn. Reson. Med. 29, 599-604 (1993); Weissleder, et al. Am. J. Roentgeneol, 152, 167-173 (1989); Krueter et al., PCT/EP2007/002198, each reference incorporated herein by reference in its entirety). Such nanoparticles can be used, for example, in clinical settings for magnetic resonance imaging of bone marrow cells, lymph nodes, spleen and liver (see, e.g., Shen et al., Magn. Reson. Med. 29, 599 (1993); Harisinghani et al., Am. J. Roentgenol. 172, 1347 (1999); each reference incorporated herein by reference in its entirety.) For example, magnetic iron oxide nanoparticles sized less than 50 nm and containing cross-linked cell membrane-permeable TAT-derived peptide efficiently internalize into hematopoietic and neural progenitor cells in quantities of up to 30 pg of superparamagnetic iron nanoparticles per cell (Lewin et al., Nat. Biotechnol. 18, 410 (2000)). Furthermore, the nanoparticle incorporation does not affect proliferative and differentiation characteristics of bone marrow-derived CD34+ primitive progenitor cells or the cell viability (Lewin et al., Nat. Biotechnol. 18, 410 (2000)). Accordingly, the disclosed nanoparticles can be used not only for in vivo tracking of the labeled cells, but can also be very useful when in vivo gene editing is used. The labeled cells retain their differentiation capabilities and can also be detected in tissue samples using magnetic resonance imaging. Disclosed herein are novel nanoparticle-based compositions, which are functionalized to carry various sets of RNA and/or DNA, proteins, peptides and other small molecules that can serve as excellent vehicles for intracellular delivery of biologically active molecules to target a specific nucleotide sequence of interest, introduce nucleotide sequence alterations of interest and thereby modulate cellular function and properties.

### General Description of Nanoparticle-Peptide/Protein/microRNA Conjugates

Nanoparticles can be core-based, such as comprising iron oxide or gold. In some embodiments, the nanoparticles can comprise or other, e.g., polymeric, material with biocompatible polymer coating (e.g., dextran polysaccharide) with X/Y functional groups, to which linkers of various lengths are attached, and which, in turn, are covalently attached to proteins, RNAs or DNAs and/or peptides (or other small molecules) through their X/Y functional groups. Linker structures are well-known and can be routinely applied to the disclosed functionalized nanoparticle design. Linkers can provide conformational flexibility to the attached bioactive compound, such as protein or polynucleotide, such that it can maintain its proper three-dimensional structure and rotate to more efficiently interact and bind with its extracellular or intracellular partner.

Illustrative, non-limiting examples of functional groups that can be used for crosslinking include:

—NH₂ (e.g., lysine, a —NH₂);

—SH;

—COOH;

—NH—C(NH)(NH₂);

-carbohydrate;

-hydroxyl (OH); and

attachment via photochemistry of an azido group on the linker.

Illustrative, non-limiting examples of crosslinking reagents include:

SMCC [succinimidyl 4-(N-maleimido-methyl) cyclohexane-1-carboxylate], including sulfa-SMCC, which is the sulfosuccinimidyl derivative for crosslinking amino and thiol groups;

LC-SMCC (Long chain SMCC), including sulfo-LC-SMCC;

SPDP [N-Succinimidyl-3-(pypridyldithio)-proprionate], including sulfo-SPDP, which reacts with amines and provides thiol groups;

LC-SPDP (Long chain SPDP), including sulfo-LC-SPDP;

EDC [1-Ethyl Hydrocholride-3-(3-Dimethylaminopropyl)carbodiimide], which is a reagent used to link a —COOH group with a —NH₂ group;

SM(PEG)n, where n=1, 2, 3, 4 . . . 24 glycol units, including the sulfo-SM(PEG)n derivative;

SPDP(PEG)n, where n=1, 2, 3, 4 . . . 12 glycol units, including the sulfo-SPDP(PEG)n derivative;

PEG molecule containing both carboxyl and amine groups; and

PEG molecule containing both carboxyl and sulfhydryl groups.

Illustrative, non-limiting examples of capping and blocking reagents include:

citraconic anhydride, which is specific for NH;

ethyl maleimide, which is specific for SH; and

mercaptoethanol, which is specific for maleimide.

The nanoparticles useful for such purposes can contain a metal core such as iron oxide or gold, or can be polymeric nanoparticles without a metal core, but containing trapped-inside or otherwise linked bioactive molecules that can be released over time, leading to alternating and/or long-lasting effects.

In view of the foregoing, we have treated biocompatible nanoparticles with functional amines on the surface to chemically bind proteins, nucleic acids and short peptides, as described in U.S. Pre-Grant Publication No. 2014/0342004, published Nov. 20, 2014, and international Application No. PCT/US2017/035823, filed Jun. 3, 2017, each incorporated herein by reference in its entirety. Briefly, the superparamagnetic or alternative nanoparticles can be less than 50 nm or larger in size and with 10 or more amine (or other) functional groups per nanoparticle.

SMCC (such as from Thermo Fisher) is dissolved in dimethylformamide (DMF) obtained from, for example, ACROS (sealed vial and anhydrous) at the 1 mg/ml concentration. Sample is sealed and used almost immediately.

Ten (10) microliters of the solution are added to nanoparticles in 200 microliter volume. This provided a large excess of SMCC to the available amine groups present, and the reaction is allowed to proceed for 1-2 hours. Excess SM and DMF can be removed using a centrifugal filter column (such as from Amicon®) with a cutoff of 3,000 daltons. Five exchanges of volume are generally required to ensure proper buffer exchange. It is important that excess of SMCC be removed at this stage.

Any RNA, DNA, or peptide-based molecule, for example, commercially available Green Fluorescent Protein (GFP) or purified recombinant GFP, or any other proteins of interest, are added to the activated nanoparticles. The bioactive molecule-nanoparticle solutions are reacted and the unreacted molecules are removed by centrifugal filter units with appropriate MW cutoff (in the example with GFP it is at least 50,000 dalton cut-off). The sample is stored at −80° C. freezer or at 4° C. Instead of using Amicon® centrifugal filter columns, small spin columns containing solid size filtering components, such as Bio Rad P size exclusion columns can also be used. It should also be noted that SMCC also can be purchased as a sulfa-derivative (Sulfo-SMCC), making it more water soluble. DMSO (dimethyl sulfoxide) may also be substituted for DMF as the solvent carrier for the labeling reagent; again, it should be anhydrous.

All the other crosslinking reagents can be applied in a similar fashion. SPDP is also applied to the appropriate protein/peptide in the same manner as SMCC. It is readily soluble in DMF. The dithiol is severed by a reaction with DTT for an hour or more. After removal of byproducts and unreacted material, it is purified by use of an Amicon® centrifugal filter column with at least 3,000 dalton MW cutoff.

Another means of labeling a nanoparticle with a peptide, DNA, RNA, or protein would be to use different bifunctional coupling reagents, as we described in U.S. Pre-Grant Publication No. 2014/0342004, incorporated herein by reference in its entirety.

### Attachment of Peptides, DNAs, RNAs and Proteins on a Nanoparticle

In one embodiment, various ratios of SMCC labeled proteins and peptides are added to the beads and allowed to react. Exemplary proteins and peptides are described in more detail below.

In another aspect, the present disclosure is also directed to methods of delivering bioactive molecules attached to functionalized nanoparticles for modulation of intracellular activity via targeted editing of a nucleotide sequence to normalize/modify a gene sequence, control expression of a gene of interest, and/or introduce a new gene for expression in the cell. For example, animal or human stem or other cell types, commercially available or obtained using standard or modified experimental procedures, are first plated under sterile conditions on a solid surface with or without a substrate to which the cells may adhere if needed (feeder cells, gelatin, martigel, fibronectin, and the like). The plated cells are cultured for a time with a specific factor combination that allows cell division/proliferation or maintenance of acceptable cell viability and concentration. Examples are serum and/or various growth factors as appropriate for the cell-type, which can later be withdrawn or refreshed and the cultures continued. The plated cells are cultured in the presence of functionalized biocompatible cell-permeable nanoparticles with covalently linked target nucleotide sequence binding and modifying factors (that include but are not limited to peptide, DNA or RNA-based guiding molecules, a bi-functional or multifunctional enzyme with binding affinity to the guiding molecules and its nuclease activity, and, optionally, a donor nucleotide sequence necessary for gene correction) attached using various methods briefly described herein and elsewhere (see, e.g., U.S. Pre-Grant Publication No. 2014/0342004, incorporated herein by reference in its entirety) in the presence or absence of magnetic field. The use of a magnet in case of biocompatible superparamagnetic nanoparticles renders an important increase in the contact surface area between the cells and nanoparticles and thereby reinforces further improved penetration of functionalized nanoparticles through the cell membrane. Furthermore, applying a magnetic field after editing a nucleotide sequence encoding the gene of interest in the cells may aid in removal of functionalized nanoparticles from the treated cells which will further minimize the off-target effects of such gene editing, thus preserving the genome integrity of the treated cells.

The cells are maintained attached or suspended in culture medium, and non-incorporated nanoparticles are removed by centrifugation or cell separation, leaving cells that are present as clusters. The cells are then resuspended and recultured in fresh medium for a suitable period. The cells can be taken through multiple cycles of separating, resuspending, and reculturing until gene editing is confirmed prior to subsequent use of the cells in vitro or in vivo. The current disclosure is applicable to introduce single or multiple nucleotide substitutions, nicks (cuts in one strand of double-stranded DNA), deletions, insertions in the gene of interest or any gene-regulatory sequence, but also for introduction of premature truncation resulting in heterozygous or homozygous knock-out of the gene of interest. A broad range of cell types can be used such as human fibroblasts, blood cells, epithelial cells, mesenchymal cells, and the like.

Gene editing is based on the treatment of various cell types or tissues with bioactive molecules that can include various polypeptides, RNA and DNA molecules. Such bioactive molecules alone do not penetrate through a cell membrane efficiently, may not reach the cell nuclei without a special delivery vehicle targeting adherent or suspension cells in vitro or in vivo. Furthermore, these bioactive molecules have a short half-life and can undergo degradation upon exposure to various proteases and nucleases on the route to a cell nucleus, which altogether will result in a low gene editing efficiency overall. These disadvantages result in reduced efficacy of the bioactive molecules, and therefore require much higher doses of a treatment to achieve a noticeable gene editing effect, which, in turn, leads to unwanted increases in off-target activity. Therefore, in the current disclosure functionalized nanoparticles are used to overcome the abovementioned disadvantages. More specifically, these bioactive molecules, when linked to the nanoparticles and compared with the original “naked” state, acquire new physical, chemical, biological functional properties that confer cell-penetrating and cell cytoplasm, nucleus or mitochondria targeting ability, larger size, altered overall three-dimensional conformation and the acquired capability to edit nucleotide sequence and/or expression of target gene(s) of interest. Since the first reports in 2013 demonstrating the suitability of the class 1 CRISPR/Cas9 nuclease system and later the class 2 CRISPR/Cpfl for gene editing in mammalian cells, many studies have been performed characterizing the mechanics and applicability of such editing systems. See, e.g., Cong et al., Multiplex Genome Engineering Using CRISPR/Cas Systems. Science 339:819, (2013); Mali et al., Cas 9 as a Versatile Tool for Engineering Biology, Nat. Methods 10, 957, (2013). A number of guiding molecules and gene products with nuclease activity have subsequently been reported to exhibit gene editing effects, and the list continues to grow (Hsu et al., Development and Applications of CRISPR-Cas9 for Genome Engineering, Cell 157, 1262 (2014); Jiang et al., Multigene Editing in the Escherichia coli Genome via the CRISPR/Cas System, Appl. Environ. 81, 2506 (2015); Doench et al., Rational Design of Highly Active sgRNAs for CRISPR-Cas9-Mediated Gene Inactivation, Nat Biotechnol. 32, 1262 (2014); Tsai et al. GUIDE-seq Enables Genome-Wide Profiling of Off-Target Cleavage by CRISPR-Cas Nucleases, Nat Biotechnol. 33, 187 (2015); Fu Y et al, Targeted Genome Editing in Human CellsU CRISPR/Cas Nucleases and Truncated Guide RNAs, Methods Enzymol. 546, 21 (2014); Wyvekens et al., Dimeric CRISPR RNA-Guided FokI-dCas9 Nucleases Directed by Truncated gRNAs for Highly Specific Genome Editing, Hum Gene Ther. 26, 425 (2015); Kim et al., Highly Efficient RNA-Guided Genome Editing in Human Cells via Delivery of Purified Cas9 Ribonucleoproteins, Genome Res. 24, 1012 (2014); Dong et al., The Crystal Structure of Cpfl in Complex With CRISPR RNA, Nature 532, 522 (2016)).

As an example, RNA-based guiding molecules with affinity to the Cas9 nuclease and different moiety homologous to the targeted nucleotide sequence of interest and cDNA. encoding the Cas9 nuclease with nuclear localization domain were introduced into the cells using electroporation or lipofection along with a template donor sequence. The guiding molecules binding the target sequence of cellular DNA and Cas9 nuclease creates a double stand break (“DSB”) (Choulika et al., Introduction of Homologous Recombination in Mammalian Chromosomes by Using the I-SceI System of Saccharomyces Cerevisiae. Mol. Cell. Biol, 15, 1968, (1995)) in the DNA at the specific position determined by the sequence of guiding RNA. Two such DSBs generate deletion of the region of interest that can be joined together by an internal mechanism of non-homologous end joining (“NHEJ”), thereby removing the nucleotide sequence of interest (Bibikova et al., Targeted Chromosomal Cleavage and Mutagenesis in Drosophila Using Zinc-Finger Nucleases, Genetics 161, 1169 (2002)). Alternatively, in the presence of exogenous donor DNA template containing the correct nucleotide sequence with flanking nucleotide sequences homologous to the gene of interest region, a homologous recombination takes place resulting in insertion of the correct nucleotide sequence in the place of newly created deletion. This is referred to as “homology-derived recombination” (“HDR”) (Chu et al., Increasing the Efficiency of Homology-Directed Repair for CRISPR-Cas9-Induced Precise Gene Editing in Mammalian Cells, Nat. Biotechnol. 2015, 33,543 (2015)).

Further variations of this gene editing approach include use of a nickase that is either an inactive nuclease (alone or fused or in combination with other bioactive molecules) that can alter target gene expression by virtue of binding to the target regulatory region of the gene and either activate or block its expression, or an active nuclease that creates single strand breaks (“SSB”), which is contrasted with the creation of DSB by Cas9. When used as a pair to target two nearby nucleotide sequences and in the presence of a donor sequence, the SSB can be repaired via HDR and exhibit lower (if any) non-specific off-target activity. The nickase can be represented by any enzyme like modified Cas9 or any fusion nickase enzyme generated by fusion of guiding molecule-binding domain of one gene (e.g., Cas9) with a nuclease domain of nickase (e.g., Fok1 nuclease) described previously. Guilinger et al., Fusion of Catalytically Inactive Cas9 to FokI Nuclease Improves the Specificity of Genome Modification, Nature Biotechnology 32, 577-582 (2014).

Because the off-target site binding of the nuclease (e.g., Cas9) is concentration dependent, a ribonucleoprotein particle (“RNP”) complex of the recombinant enzyme with guide-RNA has been generated for gene editing and can be introduced into the cells via electroporation or lipofection. As a result, the RNP can cleave the DNA and subsequently be degraded intracellularly, potentially resulting in lower off-target activity. See, e.g., the Alt-R CRISPR-Cas9 system and the Alt-R® S.p. HiFi Cas9 Nuclease 3NLS enzyme (Integrated DNA Technologies, Coralville, Iowa). However, the increased cell death and low transfection efficiency in hematopoietic cells, as well as the off-target sites are still an issue with this approach due to the continuous presence of the RNP in the cells. The use of magnetic field in the present disclosure for effective removal of non-integrating functionalized nanoparticles with active enzyme presents a unique way to rapidly withdraw the enzyme from the cells.

Alternative variations of this gene editing approach include the use of bioactive molecules with gene modifying activity. For example, acetylation of the lysine residues at the N-terminus of histone proteins removes positive charges, thereby reducing the affinity between histones and DNA. This makes RNA polymerase and transcription factors easier to access the promoter region. Therefore, in most cases, histone acetylation enhances transcription while histone deacetylati on represses transcription. Such histone acetylation is catalyzed by histone acetyltransferases (HATs), and histone deacetylation is catalyzed by histone deacetylases (HDACs). DNA methylation is the addition of a methyl group (CH₃) to the DNA's cytosine base by tnethyltransferases that affect gene transcription. The methylation pattern is heritable after cell division, hence DNA methylation plays an important role in control of cell fate during development.

Potential problems with current gene editing approaches include premature degradation of the RNP which may bind the target site but not cleave DNA due to intracellular proteolysis of the enzyme and lost nuclease activity. Such problems are addressed by the present disclosure, which, among other advantages, provides for the use of additional degradation-protecting compounds, such as a nanoparticle or a PEG or other compound or molecule functionalized in the absence of DNA with non-integrating peptides, proteins and RNA molecules, thereby preserving the cell genome intact.

Furthermore, as indicated above, the established use of lentiviral vectors for delivery of guiding molecules and nucleases inside the cells is known to result in random integration of viral DNA into the human cell genome and may lead to detrimental consequences such as cancer. The present disclosure overcomes this problem upon generation and use of the nanoparticles functionalized using abovementioned and/or other gene editing molecules as non-integrating complexes that preserve the cell genome intact.

In alternative strategies, current gene editing tools can also be based on the expression of gene products delivered to the cells using non-viral plasmid DNA. Again, any use of DNA is prone to trigger unpredictable random insertion of nucleotides into the genomic DNA of the host cell thereby potentially leading to detrimental consequences or skewing the phenotype. The present disclosure addresses this issue by presenting an innovative approach that is based on non-integrating multi-functional nanoparticles with cell-penetrating capacity with highly efficient delivery of components necessary for gene editing.

The current disclosure overcomes the insertional mutagenesis and skewing genotype/phenotype problems by using cell membrane penetrant functionalized and non-integrating nanoparticles. The nanoparticles can be metal-core (e.g., superparamagnetic iron-based (when rapid removal of nucleases using electromagnetic field is needed) or gold based nanoparticles) or non-cored (e.g., polymeric nanoparticles, such as those based as an example on PLA/PLGA, liposomes, or micelles) functionalized with any of the abovementioned or other bioactive molecules exposure to which may result in gene editing, i.e., targeted changes in the nucleotide sequence of genes of interest. The recited cell types, factors, and/or combinations of factors are not intended to be limiting and that additional factors and/or combinations will be newly discovered and that those combination would work in the same way as described in the application.

The guide nucleic acid molecule, the modifying factor (e.g., nuclease such as cas9, Cpfl, homologs or functional derivatives thereof or other proteins with various activities), and/or the donor nucleic acid molecule can all be conjugated to the same nanoparticle or alternatively, one or more of the aforementioned components can be conjugated to different nanoparticles in any combination. For example, the modifying factor (e.g., nuclease or nickase) with the guide nucleic acid molecule can be conjugated to the same nanoparticle whereas the donor nucleic acid molecule, if employed, can be conjugated to a different nanoparticle. Alternatively, the guide nucleic acid molecule and the donor nucleic acid molecule can be conjugated to the same nanoparticle whereas the modifying factor (e.g., nuclease) can be conjugated to a different nanoparticle. Alternatively, the modifying factor (e.g., nuclease) and the donor nucleic acid molecule can be conjugated to the same nanoparticle whereas the guide nucleic acid molecule can be conjugated to a different nanoparticle. As yet another alternative, each of the three components can be conjugated to separate, individual nanoparticles. In any of the foregoing embodiments, the multiple nanoparticles can all be the same or different nanoparticle types, as described in more detail above. Furthermore, the individual functionalized NPs are not aggregated together in larger constructs/complexes, but instead are separate individual functional constructs capable of penetrating through cell membrane and delivering cargo intracellularly.

The donor nucleotide sequence, if needed, can be a DNA or RNA sequence that is intended to be inserted into (or have a portion thereof be inserted into) the target DNA or RNA molecule. This is useful for various applications, as described above, such as correcting a deleterious sequence in the cell genome. Such deleterious sequence can be, for example, a mutation resulting in a negative phenotype or an exogenous sequence from a pathogen. Alternatively, the donor nucleotide sequence can include a modified sequence to affect the expression levels of a gene within the target genome. This can be, for example, providing a different or modified promoter sequence that enhances or reduces expression of the gene, but which does not otherwise modify the actual encoding sequence of the gene itself. As yet another example, the donor nucleotide sequence can introduce a heterologous encoding sequence (with or without a promoter sequence) to provide the cell the ability to express the heterologous gene and ultimately produce a new protein.

Another application of the disclosure is the screening/testing of a bioactive molecule (compound or compounds) for regulated gene editing and its expression. This involves combining the compound attached to the nanoparticle using methods disclosed herein with a cell population of interest (whether fibroblasts, blood cells, mesenchymal cells, and the like), culturing for suitable period and then determining any modulatory effect resulting from the compound(s). This includes knocking out virtually any gene product of interest, changes in nucleotide sequences of genes with one or more mutations whether those are single or multi-nucleotide substitutions, insertions, truncations or deletions to be further used for direct cell reprogramming and/or generation of specialized functional cell types of interest, such as cardiac cells, hepatocytes (liver cells), or neural cells, examination of the cells for toxicity, metabolic change, or an effect on contractile activity and/or other function.

Another use of the described compositions is the formulation of specialized cells as a medicament or in a delivery device intended for treatment of a human or animal body. This enables the clinician to administer the non-integrating nanoparticles functionalized with gene editing molecules described above or other protein or RNA based molecules in or around a tissue of interest (e.g., heart, bone marrow, brain or liver, etc.), either from the vasculature or directly into the muscle or organ wall, thereby allowing the specialized cells to engraft, limit the damage, and/or participate in regeneration/regrowth of the tissue's infrastructure and restoration of specialized function. Alternatively, the cells with an edited genome can be produced in vitro with the described functionalized nanoparticles, modified by targeted reprogramming into a special cell type of interest if needed, and administered thereafter into the area around diseased or damaged tissue of a subject.

Unless specifically defined herein, all terms used herein have the same meaning as they would to one skilled in the art of the present disclosure. Practitioners are particularly directed to Sambrook J., et al., (eds.) Molecular Cloning: A Laboratory Manual, 3rd ed., Cold Spring Harbor Press, Plainsview, N.Y. (2001); Ausubel et al., (eds.), Current Protocols in Molecular Biology, John Wiley & Sons, New York (2010).

The use of the term “or” in the claims is used to mean “and/or” unless explicitly indicated to refer to alternatives only or the alternatives are mutually exclusive, although the disclosure supports a definition that refers to only alternatives and “and/or.” The terms “gene” and “gene product” are used interchangeably.

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the terms “herein,” “above,” and “below,” and words of similar import, when used in this application, shall refer to this application as a whole and not to any particular portion of the application.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed to edit a gene of interest whether by correcting a mutation, introducing a nucleotide sequence alteration in a target gene, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited is hereby specifically incorporated by reference in their entireties.

As way of further illustration and not limitation, the following Examples disclose other aspects of the present disclosure.

### EXAMPLE 1

### Knock Out PD1 Gene with Non-Integrating Functionalized Nanoparticles

Programmed cell death protein 1, also known as PD-1 and CD279 (cluster of differentiation 279), is a protein that in humans is encoded by the PDCD1 gene. See, Shinohara T, Taniwaki M, Ishida Y, Kawaichi M, Honjo T. Structure and Chromosomal Localization of the Human PD-1 gene (PDCD1), Genomics. 1994; 23: 704-6; and the NCBI full report on PDCD1, “Programmed cell death 1 [Homo sapiens (human)]; Gene ID: 5133, updated on 8 Oct. 2017. PD-1 is a cell surface receptor, it is known to bind at least two ligands, PD-L1 and PD-L2 and functions as an immune checkpoint. PD-1 plays an important role in down regulating the immune system by preventing the activation of T-cells, which in turn reduces autoimmunity and promotes self-tolerance. The inhibitory effect of PD-1 is accomplished through a dual mechanism of promoting apoptosis (programmed cell death) in antigen specific T-cells in lymph nodes while simultaneously reducing apoptosis in regulatory T cells (suppressor T cells). See, Francisco L M, Sage P T, Sharpe A H (July 2010). The PD-1 Pathway in Tolerance and Autoimmunity, Immunological Reviews. 2010; 236: 219-42; and Fife B T, Pauken K E. The role of the PD-1 Pathway in Autoimmunity and Peripheral Tolerance, Annals of the New York Academy of Sciences, 1217:45, 2011. Therefore, a new class of drugs that block PD-1, the PD-1 inhibitors, activate the immune system to attack tumors and are thereby used with varying success to treat some types of cancer. See Schumann K, Lin S, Boyer E, Simeonov D R, Subramaniam M, et at Generation of Knock-In Primary Human T Cells Using Cas9 Ribonucleoproteins, PNAS, 112:10437-42, 2015. The non-integrating functionalized nanoparticles described above can be used to turn off (e.g., knock-out) the PD-1 gene expression in target cells as an attractive potent alternative to PD-1 inhibitors.

For attachment of Cas9 nuclease and guiding nucleic acid molecules various routes of functionalization can be used with one of such routes presented below. Nuclease Cas9 is linked to the nanoparticle (can be superparamagnetic, gold or polymeric composite nanoparticle) using LC-SMCC as the cross linker chain (LC1, attached to the amine groups of the nanoparticle), which is then coupled directly to the sulphydryl group of Cas9. LC-SMCC (from Thermo Fisher) is dissolved in dimethylformamide (DMF) obtained from ACROS (sealed vial and anhydrous) at the 1 mg/ml concentration. Sample is sealed and used almost immediately.

One (1) to ten (10) microliters of the solution are added to nanoparticles in 200 microliter volume, which provided various excess ratio of SMCC to the available amine groups present, and the reaction is allowed to proceed for one hour. Excess SMCC and DMF can be removed using an Amicon® spin filter with a cutoff of 3,000 daltons. At least five exchanges of volume required to ensure proper buffer exchange. It is crucial that excess of LC1 (SMCC) be removed at this stage. Subsequently, a cell-penetrating peptide with terminal cysteine residue (described in International Application Publication No. WO/2013/059831, incorporated herein by reference in its entirety) is allowed to briefly react with SMCC on nanoparticle and the non-bound peptide is removed by at least five washes using Amicon® spin filters described above. At this stage, some amine groups on nanoparticles will remain intact, thereby providing docking sites for covalent attachment of second different length linker chain (LC2), which is attached using the same procedure described above for SMCC. Again, it is crucial that excess of LC2 be removed at this stage.

The Cas9 or Cpfl nuclease (or other nuclease/nickase) with a free-standing cysteine is pre-incubated 10 min at 37° C. with PD-1 specific guiding RNA molecules (gRNAs) as described (Schumann K., et al., 2015) or added to a nanoparticle along with gRNAs with homology to a target sequence of PD-1 in a 1:1 ratio and the reaction is allowed to proceed for two hours at 4° C. The excess reagent is removed by passing the functionalized superparamagnetic nanoparticles using available appropriate size columns or magnet from different vendors such as Myltenyi Biotech and the resultant product is used for gene editing in vitro and in vivo.

The human primary T cells isolated either from fresh whole blood or buffy coats as described (Schumann K., et al., 2015) are treated with non-integrating cell-penetrant nanoparticle functionalized with Cas9 nuclease and target-specific gRNAs. Briefly, 100,000 cells cultured under sterile conditions on a solid surface in a humidified incubator with 5% CO₂ and ambient O₂ are treated with a suspension containing cell-permeable functionalized nanoparticles with bioactive molecules in the presence or absence of magnetic field. The functionalized nanoparticles are effective in intracellular delivery of its cargo into adherent as well as into suspension cells and do not require lipofection or electroporation.

The use of magnetic field in case of superparamagnetic nanoparticles renders an important increase in the contact surface area between the cells and nanoparticles and thereby ensuring improved penetration of functionalized nanoparticles through the cell membrane. Importantly, similar to poly(ethylene glycol) PEG-mediated protection of several protein-based drugs (PEG-GCSF, Amgen, Calif.; PEG-Interferon, Schering-Plough/Merck, N.J.) to which PEG is attached, the nanoparticles used in conjunction with coupled peptides increase the size of the polypeptide and masks the protein's surface, thereby reducing protein degradation by proteolytic enzymes and resulting in higher gene editing efficiency.

The cells are suspended in culture medium, and non-incorporated nanoparticles can be removed by centrifugation for 10 minutes at approximately 1200×g, leaving cells that are present as clusters in the pellet. The clustered cells are then resuspended, washed again using similar procedure and recultured in fresh medium for a suitable period. The cells can be taken through multiple cycles of separating by cell cloning or serial dilutions, resuspending, and reculturing in a culture media until a consequent biological effect triggered by the specific bioactive molecules delivered intracellularly is observed. It must be noted here that the Cas9 nuclease creates DSBs at its target site and the use of two different target sites in PD-1 gene ensures deletion of the PD-1 gene coding sequence with subsequent non-homologous end joining (NHEJ) repair that will result in knock-out of the PD-1 gene.

To confirm deletion of the PD-1 gene, the resultant clones are expanded and PCR is performed using genomic DNA from the cells and PD-1 specific primers across the target region for evaluation by electrophoresis on agarose gel and/or sequencing across the targeted sequence. The lack of appropriate fragment size will indicate successful knock-out of PD-1 gene. The newly generated human T-cells lacking PD-1 gene with acquired improved immunoresponsiveness can be further expanded and used for various purposes.

### EXAMPLE 2

### Inactivating PD-1 Gene Using Insertional Mutagenesis by Non-Integrating Functionalized Nanoparticles

The PD-1 gene functions via its interaction with its ligands PD-L1 or PD-L2. Hence, introducing a pre-mature stop codon within exon 1 of PD-1 will result in loss of PD-1 function in target T-cells and a significantly improved immune response due to acquired irresponsiveness to PD-1 ligand. To knock-in a premature stop codon, the functionalized nanoparticles are prepared as described above in EXAMPLE 1 except that a nickase generating a SSB instead of Cas9 (that creates a DSB) will be used along with gRNAs with homology to the target sequence in exon 1 of PD-1 gene (a pair of nanoparticles each with a nickase and different target-specific gRNA). These non-integrating functionalized nanoparticles with nickase each generates a SSB at two adjacent sites in exon 1 resulting in excision of the DNA fragment in between.

In the presence of a donor template sequence with homology to the 5′ and 3′ flanking regions of the nicked sites, a homologous recombination will take place resulting in insertion of the donor sequence with a stop codon in frame with the normal PD-1 coding sequence. To this end, a second type of cell-penetrating nanoparticle is generated by covalent attachment of modified donor DNA to LC2 site of the nanoparticle using specific procedure described above in EXAMPLE 1.

To modify DNA for linkage to LC2 of the nanoparticle, the donor DNA fragment is labeled at the 5′ end with ATPgamma-S (using commercial end-labeling DNA kit from Vector Labs, Burlingame, Calif.). The resultant modified donor DNA is suitable for subsequent covalent binding to the maleimide group of LC2 linker on the nanoparticle to be carried out as described for LC2 step in EXAMPLE 1. The type II nanoparticle with donor DNA sequence is added directly to the cell medium along with the type I nanoparticle functionalized with nickase and gRNAs and the cells are cultured and clones expanded as described in EXAMPLE 1. The clones of cells with PD-1 gene containing a premature stop codon in exon 1 are validated by PCR and agarose gel electrophoresis with PD-1 specific primers and/or by sequencing across the region of interest.

The methodologies described above can be used with nucleases and nickases, as well as with numerous DNA/RNA modifying enzymes for targeted gene editing and regulating target gene expression.

While the preferred embodiment of the disclosure has been illustrated and described it will be appreciated that various changes can be made therein without departing from the spirit and scope of the disclosure. Finally, it must be also noted that the abovementioned gene editing methodologies employing the cell membrane penetrant non-integrating functionalized biocompatible nanoparticles are applicable for editing virtually any gene of interest.

The embodiments of the invention in which an exclusive property or privilege is claimed are defined as follows:

1. A composition, comprising:
a guide nucleic acid specific for a target nucleic acid sequence,
a nuclease that modifies and/or cleaves the target nucleic acid sequence upon binding of the guide nucleic acid to the target nucleic acid sequence,
a nanoparticle, and optionally
a donor nucleic acid molecule comprising a nucleic acid sequence for insertion into the cleavage site of the target nucleic acid sequence;
wherein at least one of the guide nucleic acid and the nuclease is conjugated to the at least one nanoparticle.

2. The composition of claim 1, comprising a plurality of nanoparticles, wherein the guide nucleic acid, the nuclease, and the donor nucleic acid molecule are conjugated to the same nanoparticle or different nanoparticles in any combination.

3. The composition of claim 2, wherein the guide nucleic acid and nuclease are conjugated to the same nanoparticle.

4. The composition of claim 2, wherein the guide nucleic acid and donor nucleic acid molecule are conjugated to the same nanoparticle.

5. The composition of claim 2, wherein the nuclease and donor nucleic acid molecule are conjugated to the same nanoparticle.

6. The composition of claim 2, wherein the guide nucleic acid, nuclease, and donor nucleic acid molecule are conjugated to the same nanoparticle

7. position of claim 2, wherein the guide nucleic acid, nuclease, and donor nucleic acid molecule are each conjugated to a different nanoparticle.

8. The composition of claim 1, wherein the nanoparticle comprises at least one cell penetrating peptide (CPP) conjugated thereto.

9. The composition of claim 8, wherein the at least one CPP comprises five to nine basic amino acids.

10. The composition of claim 8, wherein the at least one CPP comprises five to nine contiguous basic amino acids.

11. The composition of claim 10, wherein the CPP comprises five to nine contiguous basic amino acids.

12. The composition of claim 1, wherein the nanoparticle has a size ranging from 1 nm to 50 nm in diameter.

13. The composition of claim 1, wherein the nanoparticle is superparamagnetic.

14. The composition of claim 1, wherein the central nanoparticle comprises iron.

15. The composition of claim 1, wherein the nanoparticle comprises a polymer coating.

16. The composition of claim 1, wherein the nanoparticle does not have a solid core.

17. The composition of claim 16, wherein the nanoparticle is polymeric, such as a liposome, micelle, and the like.

18. The composition of claim 16, wherein the nanoparticle is polymeric based on biodegradable monomers of one or more types such as PLA and/or PLGA.

19. The composition of claim 1, wherein the guide nucleic acid comprises DNA, RNA, or a combination thereof.

20. The composition of claim 1, wherein the guide nucleic acid contains sequence complimentary/homologous to the target gene sequence of interest.

21. The composition of claim 1, wherein the guide nucleic acid comprises a crRNA and a tracrRNA that are fused together.

22. The composition of claim 1, wherein the guide nucleic acid comprises a crRNA and a tracrRNA, wherein the crRNA and a tracrRNA are each conjugated to a separate nanoparticle and are allowed to associate.

23. The composition of claim 1, wherein the target nucleic acid sequence is in the genomic DNA of a cell.

24. The composition of claim 1, wherein the target nucleic acid sequence is a DNA sequence.

25. The composition of claim 24, wherein the target nucleic acid sequence is in the genomic DNA of a cell.

26. The composition of claim 1, wherein the target nucleic acid sequence is an RNA sequence.

27. The composition of claim 1, wherein the nuclease comprises a first domain that binds to the guide nucleic acid and a second domain that cleaves the target nucleic acid sequence.

28. The composition of claim 27, wherein the target nucleic acid is double stranded and the second domain cleaves the target nucleic acid to produce a double stranded break (DSB) or a single stranded break (SSB).

29. The composition of claim 27, wherein the nuclease is a fusion protein, and wherein the first and second domains are derived from distinct source proteins.

30. The composition of claim 1, wherein the nuclease comprises a functional domain of Cas9, nickase, Ago, Cpfl, or a homolog thereof.

31. The composition of claim 1, wherein the nuclease is Cas9, nickase, Ago, Cpfl, homolog thereof, or a fusion of one or more domains of any one of the foregoing nucleases.

32. The composition of claim 1, wherein the protein attached is histone deacethylase, methylase, or other proteins with one or more enzymatic activities, or a homolog thereof or a fusion of one or more domains of these proteins.

33. The composition of claim 1, wherein the composition comprises a donor nucleic acid molecule capable of homologous recombination at the cleavage site.

34. The composition of claim 28, wherein the donor nucleic acid molecule comprises sequences that can hybridize to the target sequence adjacent to the modification and/or cleavage site.

35. The composition of claim 1, further comprising a second a guide nucleic acid specific for a second target nucleic acid sequence, wherein the second target nucleic acid sequence is within 10 bases, 100 bases, 500 bases, 750 bases, 1 kb, 2 kb, 3 kb, 5 kb, 10 kb, 15 kb, 20 kb, 30 kb or more, or any number or range therein, of the target nucleic acid sequence within the same nucleic acid molecule.

36. A cell comprising the composition of any one of claims 1-35.

37. A method of altering a genome of a cell, comprising contacting the cell with the composition of any one of claims 1-35.

38. The method of claim 37, wherein the nanoparticle is magnetic and the method further comprises applying a magnetic field to the cell.

39. A method of altering a genome or transcript of a cell, comprising:
contacting the cell with one or more functionalized nanoparticles that is/are conjugated to:
a guide nucleic acid specific for a target nucleic acid sequence in the genome or transcript,
a protein capable of modifying the target nucleic acid sequence upon binding of the guide nucleic acid to the target nucleic acid sequence, and optionally
a donor nucleic acid molecule comprising a nucleic acid sequence for insertion into the cleavage site of the target nucleic acid sequence.

40. The method of claim 39, wherein the protein methylates the target nucleic acid sequence.

41. The method of claim 39, wherein the protein is a nuclease that cleaves the target nucleic acid sequence upon binding of the guide nucleic acid to the target nucleic acid sequence.

42. The method of claim 39, wherein the nanoparticle is magnetic and the method further comprises applying a magnetic field to the cell.

43. The method of claim 39, wherein the one or more nanoparticles comprise at least one cell membrane penetrating peptide (CPP) conjugated thereto.

44. The method of claim 39, wherein the guide nucleic acid, the nuclease, and the donor nucleic acid molecule are conjugated to the same nanoparticle or different nanoparticles in any combination.

45. The method of claim 37 or claim 39, wherein the cell is contacted in vitro or in vivo.
